(19) United States
(12) Patent Application Publication
(10) Pub. No.: US 2006/0224170 A1
(43) Pub. Date: Oct. 5, 2006

(54) Surgical marker clip and method for cholangiography

(76) Inventor: Michael Duff, Osage Beach, MO (US)

Correspondence Address: LATHROP & GAGE LC, 1845 S. NATIONAL, P.O. BOX 4288, SPRINGFIELD, MO, 65101, US

(21) Appl. No.: 11/394,050

(22) Filed: Mar. 30, 2006

Related U.S. Application Data
(60) Provisional application No. 60/666,535, filed on Mar. 30, 2005.

(51) Int. Cl. A61B 17/08

(52) U.S. Cl. 606/157

(57) ABSTRACT

A surgical marker clip and method for enhancing the safe performance of a cholangiography and cholecystectomy is disclosed. The clips are configured to frictionally engage the outer surface of the duct and are retained in place by light clamping force without damaging the duct. Placement of the clips allows a physician to visually isolate the common bile duct from the cystic duct during laparoscopic procedures which reduces bile duct injury typically caused by misidentification or visual misperception of the anatomy during the procedure.

### RELATED APPLICATIONS

This application claims the priority to U.S. Patent Application Ser. No. 60/666,535 filed Mar. 30, 2005, the disclosure of which is incorporated herein by reference.

### BACKGROUND OF THE INVENTION

One of the most commonly performed elective abdominal surgical procedures is the cholecystectomy for the removal of the gallbladder. It is estimated that more than 750,000 cholecystectomys are performed a year in the United States alone. Because this procedure is commonly performed laproscopically, surgeons often depend on preoperative imaging of the surgery site. A common technique is the use of intraoperative cholangiography which involves the injection of radiographic contrast material through the gall bladder into the cystic duct. The contrast material facilitates visual evaluation of the common bile duct. Injury to the common bile duct during these laparoscopic procedures is one of the leading medical malpractice claims against general surgeons.

Injuries to the common bile duct are typically caused by surgical misperception or the misidentification of the common bile duct as the cystic duct. Medical studies show that common bile duct injury in as many as 97 percent of all cases is due to visual perception illusion. (Lawrence Way Study 2002).

Common bile duct injuries often occur both during intraoperative cholangiography and during the cholecystectomy procedure. The cholangiography is typically performed to heighten the identification and perception of the surgical anatomy to decrease injuries during the laparoscopic procedure. Unfortunately, injuries can also occur during the cholangiography procedure itself since that is also performed laproscopically.

Many times, injuries to the common bile duct are due to difficulties visualizing the distinct ducts during laparoscopic dissection. Appropriate dissection takes place in the triangle of Calot within which the common bile duct and cystic duct are sometimes difficult to distinguish. Cholangiography is particularly important during laparoscopic cholecystectomy when difficulties are encountered in mobilizing (or immobilizing) the infundibulum of the gallbladder or in identifying the cystic duct, or when the surgeon suspects the presence of anatomic anomalies such as accessory or aberrant ducts.

It is believed that the significant number of injuries to the common bile duct during cholangiography or cholecystectomy procedures could be reduced by clearly distinguishing and identifying the common bile duct and the cystic duct prior to undertaking laparoscopic procedure. Once dissected and identified, it is particularly important to continue the visual disassociation of the two ducts to prevent incorrect needle placement during the cholangiography, unnecessary dissection or cutting the common bile duct due to misidentification.

At this time, the intraoperative cholangiography has been determined to be an effective method for visually identifying the hepatic duct system during cholecystectomy. A cholangiography is performed by placing a catheter through the gall bladder and into the cystic duct for the rapid introduction of a contrast material into the duct system. A variety of surgical clips and surgical clamps have been developed to frictionally and compressingly engage and hold the catheter into place within the cystic duct during the procedure. Generally, the ligating clips encircle and compress the outer wall of the cystic duct which, in turn, compresses the inner wall of the cystic duct to snugly grip and secure the cholangiography catheter.

Several problems are known to exist with the currently available clips. Generally, such clips are not radiopaque, can be difficult to place and difficult to remove. Because the clips compress the duct tissue, they can cause injury, including rips and tears, to the duct itself. Moreover, the placement of the ligating clips to secure the catheter within the duct are generally placed superior the triangle of Calot. This placement does not always allow visual isolation of the cystic duct from the hepatic duct during the cholecystectomy.

### SUMMARY OF THE INVENTION

In accordance with the present invention as embodied and described herein, a surgical marker clip is provided which, upon placement about the cystic duct prior to cholangiography or cholecystectomy creates a radiopaque marker and identifier for the visual distinction of the common bile duct from the cystic duct, or other ducts. The marker clips are sized to the duct to be marked and are removably fastened about the outer wall of the duct with enough compression for frictional retention, but not so much compression so as to cause harm or injury to the duct being marked. The clips are preferably radiopaque so they can easily be visualized post-cholangiography on x-rays. It is also preferable to use two clips spaced apart to define the work area on the cystic duct for the cholecystectomy.

In one embodiment of the inventive clip, the clip further comprises two opposing arms which are connected at a single pivot. On the first side of the pivot each arm is formed into a semi-annular extension such that when the clip is placed about the duct the arms substantially encircle the duct. The second end of the clip has a scissor-like configuration oriented about the pivot and preferably is biased with a small spring mounted between the arms at the second end. Compressingly grasping the second end opens the first end of the clip for placement around the duct and, upon releasing the second end the first ends substantially close about the duct. To remove the clip, a small tool may be used to compress the second end inward which results in the first end opening. Clips of various sizes may be provided to accommodate various duct sizes so as to ensure that the arms snugly encircle the duct without damaging it.

In a second embodiment of the invention, the clip comprises a simple compressingly fit spring member. This clip has spaced apart jaws which are generally open before it is positioned about the duct to be marked. The jaws each have distal ends which are spaced opposite one another with each jaw having a facing surface which is generally semi-circular in cross-section and concave. The marker clip is positioned by passing the duct to be marked between the spaced apart jaws and substantially encircling the duct by biasing the jaws together with compressing force. While similar clips may be used with existing applicator tools, a particular inventive tool, designed by the inventor herein is preferably used. The described clip, when used in the preferred applicator tool, is not compressed in the known clamping manner which results in a flattened clip which may damage duct tissue. Rather the clip when properly applied, is formed into a diamond shape upon closure, such that the inner surface of each of the four “legs” of the diamond engages the duct to which the clip is applied.

Also disclosed and claimed is a method for applying the clips to the cystic duct prior to performing a cholangiogram or cholecystectomy to provide a visual indicator of the duct upon which work is to be performed.

### BRIEF DESCRIPTION OF THE DRAWINGS

FIG. 1 is a perspective view of one embodiment of the inventive clip.

FIG. 2 is a perspective view of a clip in place on a duct.

FIG. 3 is a perspective view of a second embodiment of the inventive clip prior to application onto a duct.

FIG. 4 is a perspective view of the second embodiment of the inventive clip in an orientation as applied to a duct.

FIG. 5 is a perspective view of a prior art clip in an orientation as applied to a duct.

FIG. 6 is a partial view of the ducts as marked during the inventive method.

FIG. 7 is a partial view of the ducts after closure for cholestectomy.

FIG. 8 is a partial view of a tool that for applying the inventive clips.

### DETAILED DESCRIPTION OF PREFERRED EMBODIMENTS

The present invention relates to a surgical marker clip for securing about a duct prior to performing laparoscopic procedures such as cholangiography or cholecystectomy.

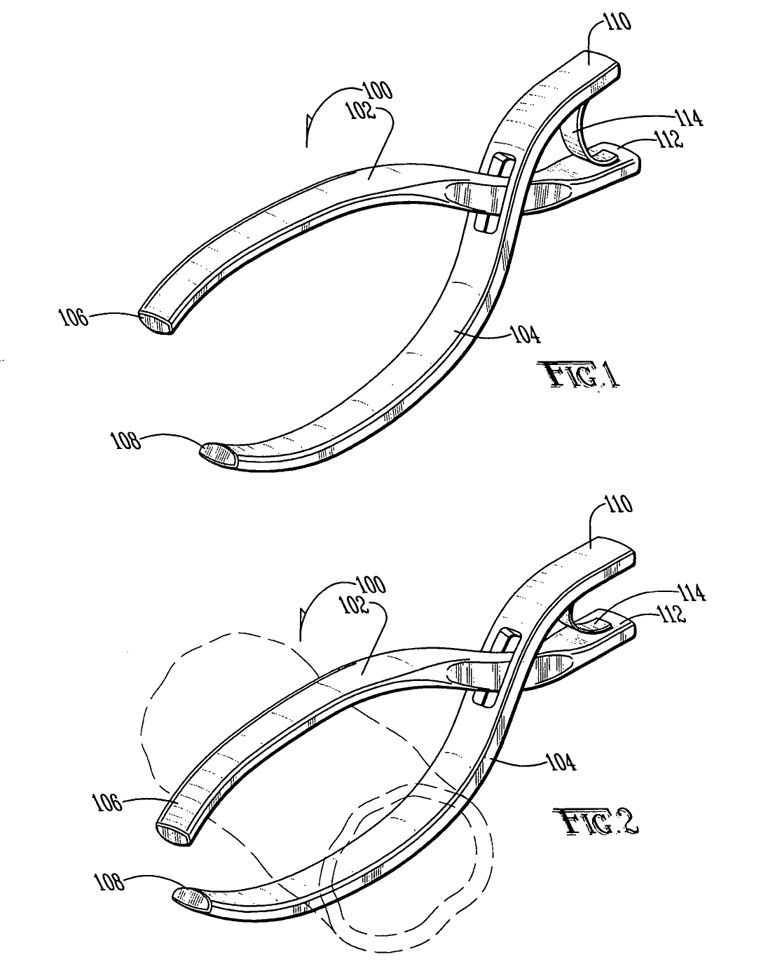

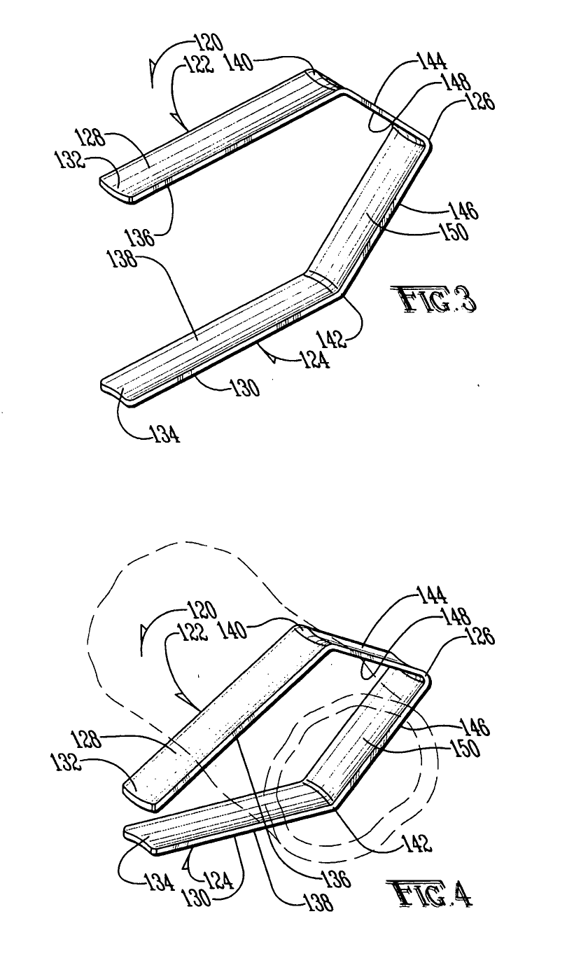

As shown in FIGS. 1 and 2, a first embodiment of the marker clip 100 has two opposed arms 102 and 104 axially displaced from each other, and further having first ends 106 and 108, second ends 110 and 112 and being further pivotally connected intermediate the first ends 106, 108 and second ends 110, 112.

A spring 114 may be seated between the second ends 110 and 112 of the clip 100 so that inward compression of the second ends 110 and 112 compresses the spring 114 and causes the first arms, 102 and 104 to move outward away from each other somewhat akin to the actuation of scissors. The first arms 102 and 104 both have generally half-round or semi-annular sections configured to snugly fit about the outer wall of a duct without compressing the duct tissue as best shown in FIG. 2.

It is preferable that the clip can be operated using commercially available surgical instruments. The marker clip described above can be operated using available laparoscopic hemostats or other instruments capable of grasping and compressing the second ends 110 and 112 of the clip. The clip is preferably radio-opaque so that it can be visualized in x-rays. Metals such as Silver or Titanium are appropriate, although other materials could be used.

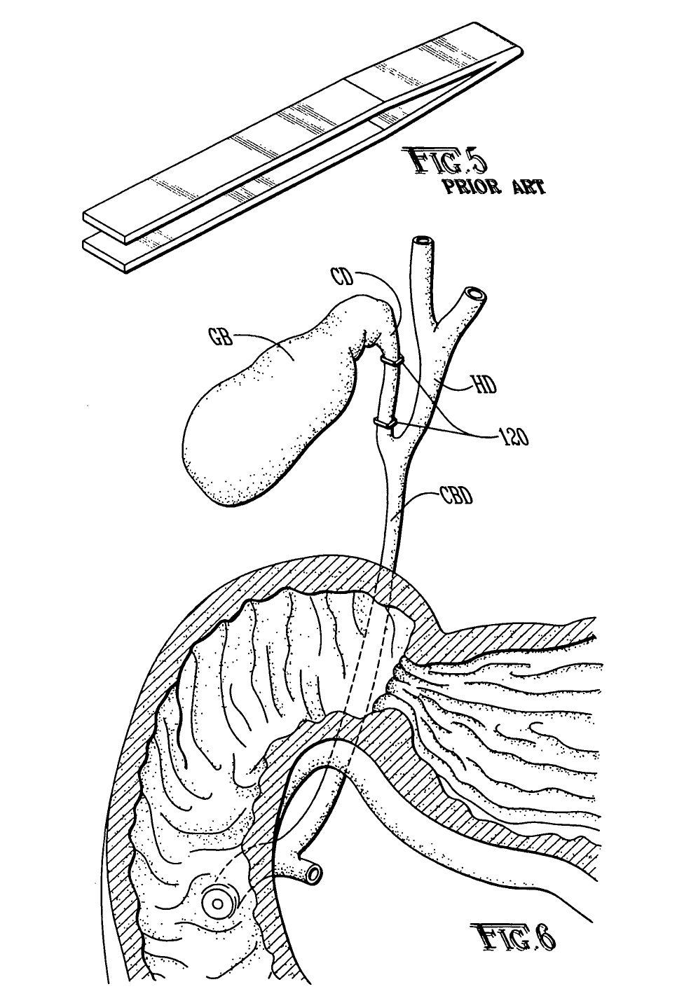

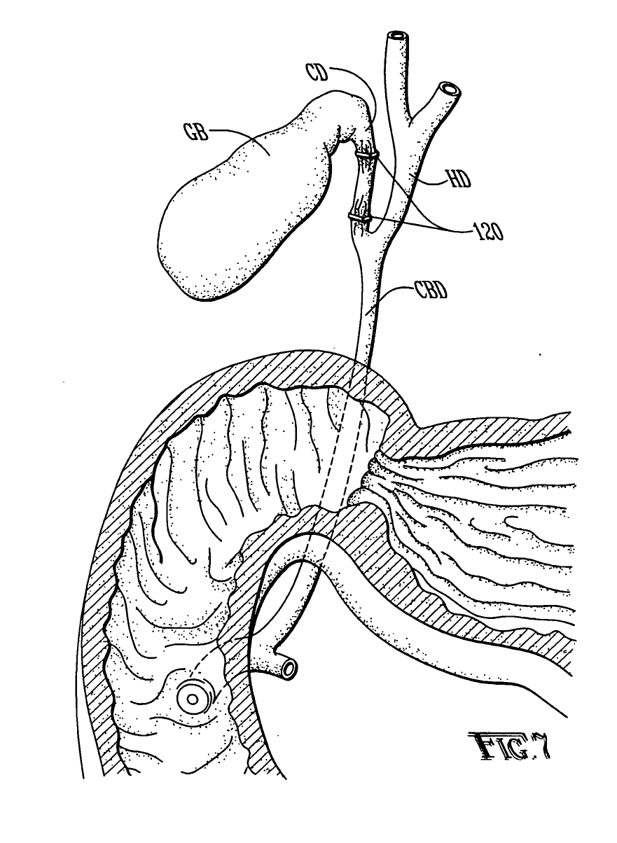

In a second embodiment of the invention, the clip 120 comprises a simple compressingly fit member as shown in FIGS. 3 and 4. This clip 120 has a first leg 122 and a second leg 124 with an apex 126 therebetween. At the terminal end of the legs 122, 124, are spaced apart jaws 128, 130 which are generally open before it is positioned about the duct to be marked. The jaws 128, 130 each have distal ends 132, 134 which are spaced opposite one another with each jaw having a facing surface 136, 138 which may be configured to provide a grasping surface to increase frictional engagement with the outer surface of the duct to which it is applied. The marker clip 120 is positioned by passing the duct to be marked between the spaced apart jaws 128, 130 and substantially encircling the duct by biasing the jaws 128, 130 together with compressing force. The tensile of the marker clip 120 allows it to be compressed about the duct without crushing or overtly compressing the duct walls. It is preferred that each leg 122, 124 is provided with a bend 140, 142 which creates an obtuse angle between the apex 126 and each jaw 128, 130. The leg segments 144, 146 between the apex and the bends 140, 142 within the leg is also provided with an inner facing surface 148, 150. Accordingly, the preferred clip, when closed, forms a diamond shape in that a geometric figure having four legs and four angles, two being obtuse and two being acute is formed. The clip 120 is maintained in place after compressed by frictional engagement of the facing surfaces 136, 138, 148 and 150 to the outer wall of the duct. The previously available clip, as shown in FIG. 5, is a generally V-shaped clip with two legs that are forcibly crushed about the duct, and as previsouly described, often damages the duct.

Application of this clip requires the use of a special clip applier pincer instrument as invented by the applicant herein and as generally shown in FIG. 8. The applier pincer instrument causes the clip 120 to be positioned about a duct to be marked and upon actuation of the instrument, the jaws 128, 130 are forcibly urged together and the inner facing surfaces 136, 138, 148, 150 are seated against the duct wall. The tool applies inward closing pressure on the bends 140 and 142 which results in the closure of the distal ends 132, 134 of the jaws 128, 130. The clip 120 is compressed only as much as is necessary for the clip to be frictionally retained on the duct. There is no deformation or degradation of the duct by the force of clip attachment.

Once a cholangiography is performed so that the duct system of the gall bladder are readily identifiable, the clips 120 may be crushed using common pliers-like surgical instruments so that the cholecystectomy can be performed between the marker clips 120, as more specifically described herein These clips are also radiopaque so that they can be visualized in x-rays.

These clips are not intended to be ligating clips which can be used to retain a catheter within the duct, such as the clip shown in FIG. 4 which is in the closed or retention position, although the clips certainly could be used to perform that function. As can be seen, the compression of the clip would significantly close the lumen of the duct to which it is applied which is a downfall with existing clips.

As shown in FIG. 6, a method for marking the hepatic duct HD for visual identification to reduce or eliminate a common bile duct CBD injury during cholecystectomy is also claimed and comprises the steps of: visually identifying the gallbladder GB, visually identifying and separating the cystic duct CD from the hepatic duct HD, visually identifying and isolating the common bile duct CBD, applying at least one and preferably two of the inventive marker clips 120 around the cystic duct CD for visual identification in a spaced apart orientation to define a work space along the cystic duct CD, and performing a cholangiogram by inserting a percutaneous cholangiogram catheter through the gallbladder GB and into the marked cystic duct CD. Once the cholangiogram is performed the marker clip 120 should be identifiable on the cystic duct CD rather than the hepatic duct HD or common bile duct CBD.

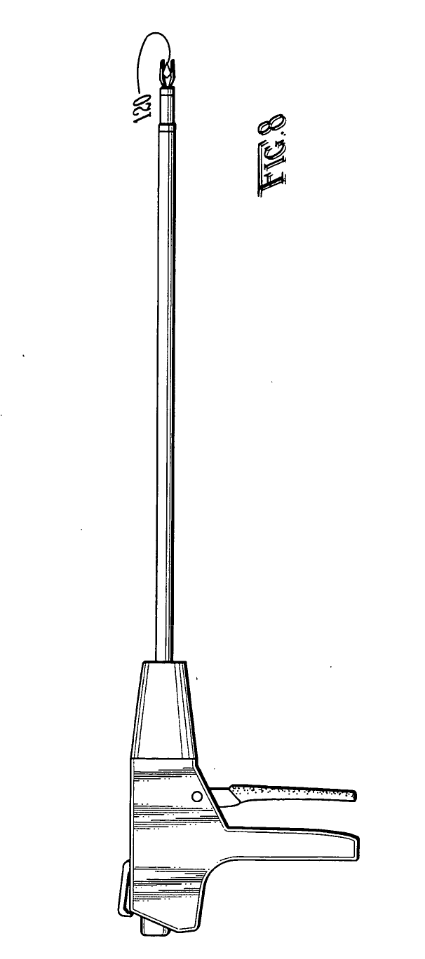

To then perform a cholecystectomy, the marker clips 120 can then be fully compressed to substantially close the cystic duct as shown in FIG. 7. The cystic duct is then cut or divided between the marker clips. Placement of the marker clips on the cystic duct prior to performing the cholangiogram and then visually identifying that the marker clips are appropriately placed on the cystic duct as opposed to the common bile duct or hepatic duct ensures that the cholecystectomy is performed on the correct duct. The marker clips essentially define a “safety zone” which, when visually identified on the cholangiogram gives confidence that the appropriate duct has been identified and will be cut during the cholecystectomy.

It is to be understood that while certain embodiments of the present invention have been illustrated and described herein that such is not to be limiting. There are many changes and modifications which can be made to applicant's device and inventive procedure which are intended to be included within the scope of applicant's invention. It is intended that applicant invention be limited only by the scope of the claims appended hereto.

What is claimed is:

1. A duct marker clip for releasably securing to a duct during surgical laparoscopic procedures for visual identification of a work area, comprising a first arm and a second arm, each arm having a first end and second end and wherein the arms are pivotally connected intermediate the first end and second end, and wherein the first and second arm at the first end each are curved generally inward, with each arm having inner surfaces for frictionally engaging the duct.

2. The duct marking clip of claim 1 further comprising a spring member mounted between the second ends of the first and second arms such that when pressure is applied generally inwardly thereto the first ends open outward and when the second ends are released the spring biases the second ends outward and the first ends inward.

3. The duct marking clip of claim 1 wherein the first and second arms are malleable so that they can be forcibly compressed inwardly to frictionally engage an outer wall of a duct to be marked and upon further forcible compression cause substantial closure of the duct.

4. The duct marking clip of claim 1 wherein the first and second arms are each substantially C-shaped and wherein both of the arms are malleable so that they can be compressed inwardly to encircle and frictionally engage an outer wall of a duct to be marked.

5. The duct marking clip of claim 1 wherein the first and second arms each have at least one obtuse angle provided therein such that upon closure of the clip, a diamond shaped cross section is formed.

6. A duct marking clip comprising a first leg connected at an apex to a second leg, each leg having a jaw end and a bend intermediate the apex and the jaw end, such that the bends are oriented generally outward from the apex and the jaw ends and accordingly the clip has a substantially diamond shaped cross section.

7. The duct marking clip of claim 6 wherein each leg is provided with inner facing surfaces which frictionally engage the outer surface of the duct to be marked to retain the clip in place.

8. The duct marking clip of claim 6 wherein the first and second legs are malleable so that they can be forcibly clamped inward to compress the clip onto the duct.

9. The duct marking clip of claim 8 wherein the clip is compressed inward to frictionally secure the clip onto the duct by exerting generally inward force on the bend formed in each leg.

10. A method for visually identifying a duct during a laparoscopic cholecystectomy, comprising the steps of: identifying and dissecting the cystic duct; marking the cystic duct by placing at least two marking clip around the outer wall of the cystic duct to define a work area; attenuating the gallbladder percutaneously with a needle, placing a catheter through the gallbladder into the cystic duct; injecting a contrast into the gallbladder and cystic duct; visually identifying the defined work area on the cystic duct; and visually discerning the cystic duct from the common bile duct or hepatic duct.

11. The method of claim 10 further comprising the step of performing a cholecystectomy after visually discerning the cystic duct from the common bile duct and hepatic duct by cutting the cystic duct within the defined work area.
